(19) United States
(12) Patent Application Publication
(10) Pub. No.: US 2018/0133044 A1
(43) Pub. Date: May 17, 2018

(54) FLEXIBLE TOE WRAPS

(71) Applicant: PROFOOT, Inc., Brooklyn, NY (US)

(72) Inventor: Daniel FELDMAN, Brooklyn, NY (US)

(21) Appl. No.: 15/572,887

(22) PCT Filed: May 11, 2016
(86) PCT No.: PCT/US16/31803

§ 371 Date: Nov. 9, 2017

Related U.S. Application Data
(60) Provisional application No. 62/160,714, filed on May 13, 2015.

(51) Int. Cl. A61F 5/01; A61F 13/06

(52) U.S. Cl. CPC A61F 5/019; A43B 7/14; A61F 13/068

(57) ABSTRACT

A flexible toe wrap is provided to restrain at least one toe in a predetermined position for an extended period of time. The device can have a flexible body composition forming a first loop at a first end, said first loop having a diameter sized to at least allow a toe to extend therethrough; a second end of said body forming a strap having a distal end having a fastener to attach to an exterior surface of said first loop; and wherein said first loop is configured to extend distally beyond an end of the toe to form a toe pad. Preferably, the fastener is a hook and loop fastener.

### FIELD

Flexible toe wraps are described herein and, in particular, flexible toe wraps having extended toe pad features.

### BACKGROUND

Various deformities can occur to a person's toes, such as an abnormal curling or buckling of one of the lesser toes (i.e., toes 2-5) or an angular toe extension. Such deformities are commonly called a “hammer” toe. A hammer toe condition can be congenital or the result of irregular mechanics of the foot, poor footwear, arthritis, and the like. Most commonly the second toe is involved.

A hammer toe deformity can be painful, especially if shoes rub on the extended knuckle of the affected toe. Also, the skin around the extended knuckle of the toe is stretched and can break resulting in sores or it can become calloused forming what is commonly known as a ‘corn’. A hammer toe deformity can limit footwear options given the additional space needed in the toe box of an enclosed shoe to accommodate the angular flex of the deformed toe. Pain and corns can also arise at the end of the toe when the toe end rubs the ground or insole of a shoe. Pain can also occur on the side of the toe when the prominent bony areas of two toes can rub together. Furthermore, hammertoes can be associated with pain at the bottom of the ball of the foot, a condition called metatarsalgia—particularly the second toe region in association with a condition called second metatarsophalangeal stress syndrome.

Treatment of hammer toes falls into non-surgical and surgical options. Non-surgical treatment typically does not correct the deformity. The goal of non-surgical treatment is to eliminate pain and/or prevent progression of the deformity or even a reduction in the deformity. The goal of surgical treatment is to eliminate pain and correct the deformity. There are a number of different procedures that are used for the surgical correction of hammertoes. Surgical correction involves both, rebalancing the tendons and ligaments of the joint where the toe joins the ball of the foot and straightening and fusion of the joint in the middle of the affected toe. A conservative approach for the treatment of a hammer toe is to restrain the toe in a normal (or relatively normal) position and hold it in that position for a significant period of time using a toe splint. Restraining a toe postoperatively can also aid in recovery from a surgery. Toe splints and other toe restraining devices are known in the art. See generally, U.S. Pubs. 2004/0019308 to Chow and 2013/0079694 and a flexible toe wrap sold under the trade name TOE STRAIGHT by ProFoot Inc. of Brooklyn, N.Y. Despite these advances in the art, further advances are possible and desired.

### SUMMARY

Flexible toe wraps are described herein and, in particular, flexible toe wraps are provided having extended toe pad features. The toe wraps described herein can be advantageously manufactured from a single portion of material, such as a web of flexible film. The embodiments can be easily placed on a user's toes and may provide non-surgical toe straightening or straight positioning.

The toe wraps described herein may include a flexible loop and strap combination configured for retaining at least two toes in a normal position for an extended period of time. The present devices can be worn typically for up to 18 hours. Most commonly, the present embodiments would apply the loop to a user's second toe and then extending the strap under the user's third toe and around third toe circumferentially until extending over the loop on the portion atop the user's second toe where the distal end of the strap is variably and releaseably fastened to the loop, thus forming a second loop for the third toe. Preferred features of the present embodiments include extending the loop portion distally to the toe to cover the end of the user's toes. An optional embodiment also extends the loop portion proximally to the toe (i.e., towards the ball of the foot).

In one form the flexible toe wrap can include a device to restrain at least one toe in a predetermined position for an extended period of time. The device can have a flexible body composition forming a first loop at a first end, said first loop having a diameter sized to at least allow a toe to extend therethrough; a second end of said body forming a strap having a distal end having a fastener to attach to an exterior surface of said first loop; and wherein said first loop is configured to extend distally beyond an end of the toe to form a toe pad. Preferably, the fastener is a hook and loop fastener.

According to one approach, the device is formed from a single piece of material and wherein the first loop is formed by heat sealing the first end to a mid-portion of the body.

In an alternate embodiment, the toe pad extends to allow covering the toe in a direction longitudinal to the foot, the toe pad having a distal end having a fastener to fasten to the exterior surface of the device. Optionally, the toe pad can be split to form two straps, on for each toe. In another approach the first loop is configured to also extend proximally behind the toe to the ball of the foot region.

The first loop can be configured to apply to a deformed toe and allowing the distal end to extend under and around an adjacent toe, the distal end fastenable to the exterior surface of the first loop, thereby forming a second loop.

The flexible body can be a laminate comprising a soft exterior layer, a foam inner layer and a moisture resistant interior layer.

A method of restraining at least one toe in a predetermined position for an extended period of time using the present embodiments can include inserting a first toe into a first loop of a flexible body first loop at a first end, said first loop having a diameter sized to at least allow a toe to extend therethrough and said first loop is configured to extend distally beyond an end of the toe to form a toe pad; wrapping under and around an adjacent toe a second end of said flexible body forming a strap having a distal end having a fastener to attach to an exterior surface of said first loop; and attaching the fastener to the exterior surface of the first loop,

Other features will become more apparent to persons having ordinary skill in the art to which the package pertains and from the following description and claims.

### BRIEF DESCRIPTION OF THE DRAWINGS

FIG. 1 illustrates a front perspective view of an exemplary right foot toe wrap having an unassembled extendable toe pad, the left foot toe wrap being a mirror image thereof;

FIG. 2 illustrates a front perspective view of an exemplary toe wrap having an assembled extendable toe pad;

FIG. 3 illustrates the toe wrap of FIG. 2 applied to a user's foot.

FIG. 4 is a top plan view of the toe wrap of FIG. 2.

FIG. 5 illustrates a front perspective view of the toe wrap of FIG. 2 ready to apply to a user's toes.

FIG. 6 illustrates a front perspective view of the toe wrap of FIG. 2 as it would appear applied to a user's toes as shown in FIG. 3.

FIG. 7 is a top plan view of a right foot toe wrap of the present embodiments according to another approach, the left foot toe wrap being a mirror image thereof.

FIG. 8 is a top plan view of a right foot toe wrap of the present embodiments according to another approach, the left foot toe wrap being a mirror image thereof.

FIG. 9 is a front perspective view of the toe wrap of FIG. 8 as it would appear partially applied to a user's foot.

FIG. 10 is a front perspective view of the toe wrap of FIG. 8 as it would appear applied to a user's foot.

FIG. 11 is a front perspective view of a right foot toe wrap of the present embodiments according to another approach as it would appear partially applied to a user's foot, the left foot toe wrap being a mirror image thereof.

FIG. 12 is a front perspective view of a toe wrap of FIG. 11 as it would appear applied to a user's foot.

FIG. 13 is a top plan view of a universal toe wrap of the present embodiments according to another approach.

FIG. 14 is a set of toe wraps according to the embodiment illustrated in FIG. 7.

FIG. 15 is a top plan view of a universal toe wrap of the present embodiments according to another approach.

FIG. 16 is a top plan view of a toe wrap according to another approach applied to a user's foot.

FIG. 17 is a front perspective view of the toe wrap embodiment of FIG. 16.

FIG. 18 is a cross sectional view of the toe wrap of FIG. 13 taken along section lines XVIII-XVIII.

FIG. 19 is a cross sectional view of the toe wrap of FIG. 16 taken along section lines XIX-XIX.

### ELEMENT LIST

20 toe wrap
22 body
24 hook and loop fastener
26 weld position
27 distal hook portion of strap 36
28 second hook and loop fastener
30 third hook and loop fastener
32 fourth hook and loop fastener
34 extended toe pad
36 hook strap
38 toe wrap loop
39 toe wrap loop dimension
40 interior layer
42 foam layer
44 loop exterior to loop layer 38
46 loop patch
48 second loop over third toe
50 third loop over the toes rearwardly
52 optional partition of 20h

### DETAILED DESCRIPTION

Flexible toe wraps are provided as described herein and as illustrated. The present embodiments provide a means to restrain at least one toe in a normal (or near normal, or predetermined) position for an extended period of time, such as for up to 18 consecutive hours. The following embodiments may provide for reduction in pain associated with hammer toes or for some degree of realignment of deformed toes. The present embodiments may also aid in recovery from a surgery to correct a hammer toe by maintaining a desired positioning of the corrected toe. However, the present embodiments are not intended for use as a bandage or covering for open sores related to these conditions or corrections. The present toe wraps may be described and illustrated for one foot, such as a right foot. It is noted though that this description could also apply for the other foot, such as the left foot. In that case, the toe wrap for the other foot being the mirror image thereof. The present embodiments are primarily described as being applied to a user's toes. It is noted that though that the present embodiments may also be applied to a user's fingers or even any digit of any animal.

Accordingly, the present embodiments provide a flexible loop and strap combination configured for retaining at least two toes in a normal position for an extended period of time, such as up to 18 consecutive hours. Most commonly, the present embodiments would apply the loop to a user's second toe and extending the strap under the user's third toe and around third toe circumferentially until extending over the loop on the portion atop the user's second toe where the distal end of the strap is variably and releasably fastened to the loop, thus forming a second loop for the third toe. The present embodiments can be made from various soft foams suitable for skin contact such as an ethylene vinyl acetate (EVA) foam. The foam can have a thickness in the range of about 0.5 to 6 mm, but is preferably about 2-4 mm in thickness. As described herein, other layers can be laminated to the foam to provide additional strength as well as moisture and odor control. A gel layer can also be added for additional comfort. In some embodiments the loop is formed by a heat seal, particularly for the loop for the second toe, though it is noted that the loops may be formed by several fastening means such as pressure sensitive adhesives or hook and loop fasteners.

Additional features of the present embodiments include extending the loop portion distally (and alternately even onto the strap portion) relative to the toe to cover the end of the user's toes wrapped in the toe wrap. This feature provides additional comfort and may reduce corns that can also arise at the end of a hammer toe when it is rubbing on the ground or shoe. An optional embodiment also extends the loop portion proximally relative to the toe (i.e., towards the ball of the foot). This feature adds additional comfort to the ball of the foot and reduce with pain associated at the bottom of the ball of the foot of an individual with a hammer toe, called metatarsalgia. An added advantage of this optional feature is that this can eliminate the need to provide mirror image products for the left and right foot.

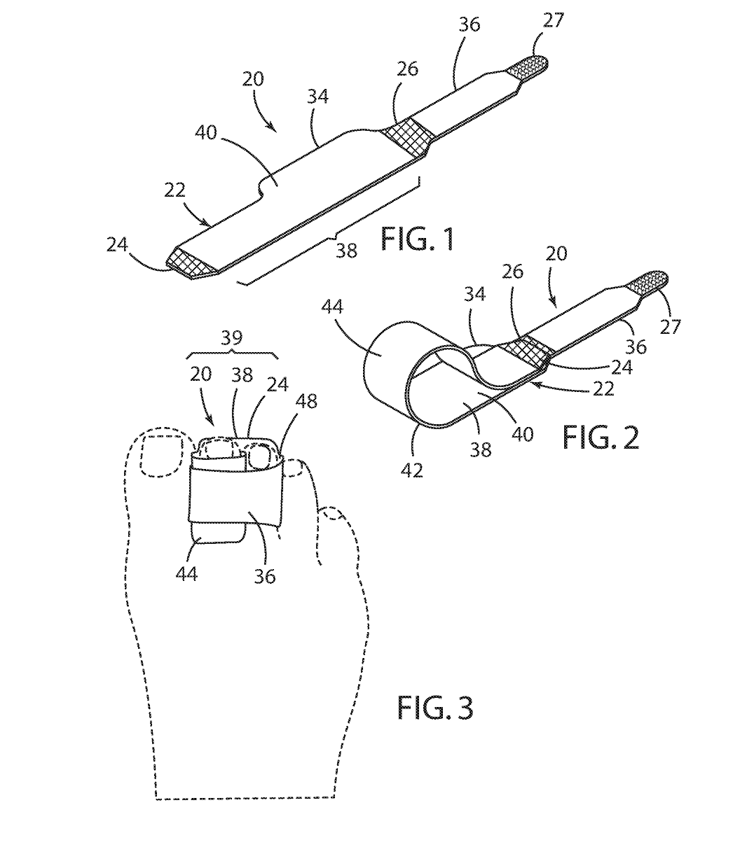

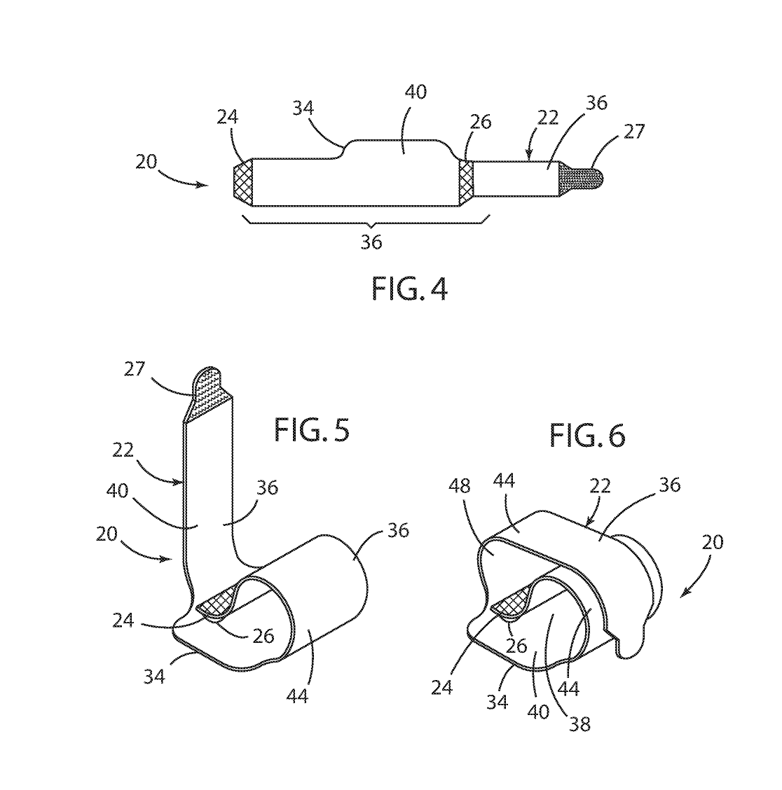

Turning now to the figures, one approach to the present embodiments is generally indicated at 20 and is shown as a body 22 in FIGS. 1-6 and being formed from a single sheet of flexible material, such as an EVA foam. A loop 38 can be form by fastening portion 24 to portion 26. In one embodiment, portion 24 is fastened to portion 26 by a permanent heat seal. In other embodiments one or both of portions can have a pressure sensitive adhesive lamination to form loop 38. Alternately, portions 24 and 26 can be a hook and loop configuration to form loop 38. Interior portion 40 of loop 38 can have a gel or moisture barrier lamination. Loop 38 is sized to at least allow a user's toe to extend therethrough, but as shown in FIG. 3, loop 38 is sized to allow it to extend under the adjacent toe when placed under the adjacent toe (see dimension 39, FIG. 3). Preferably, a portion of loop 38 is configured to extend beyond the end of a user's toes when applied forming an extended toe pad 34. Preferably, toe pad 34 can extend in the range of about 5 mm to about 20 mm (preferably about 3 mm to 8 mm) beyond the remaining portion of loop 38. It is noted though that toe pad 34 is sized so that it extends about 3-8 mm beyond the end of a user's toe when applied.

The portion of the toe wrap body 22 not enclosed in loop 38 forms a strap portion 36. The distal end 27 of strap 36 has an attachment means to attach to the exterior portion 44 of loop 38. This attachment means can be a pressure sensitive adhesive, but is preferably a hook portion of a hook and look fastened. In this instance, exterior portion 44 of loop 38 is a loop layer to allow attachment of the distal end hook portion 27 of strap 36.

FIGS. 5 and 6 show the stages of applying the toe wrap 20 as it would appear on a user's foot (not shown). In use, the user's toe (most likely the second toe) would be inserted into loop 38 so that portion extended toe pad 34 extends beyond the user's second and third toes (such as shown for a right foot in FIG. 3). Strap 36 would be wrapped around the user's third toe circumferentially and transverse to the extension of the toe forming a second loop 48. Strap 36 extends until reaching and attaching to the exterior layer 44 of loop 38. The point of attachment portion 24 of strap 36 can be anywhere on the exterior surface 44 of loop 38 based on the size of the user's toes, desired comfort and desired immobility, position retention, or realignment of the second toe. The reverse (mirror) image configuration could be performed to the left foot. Alternatively, the third toe could extend through loop 38 while the second toe is wrapped by strap 36 to form the second loop 48.

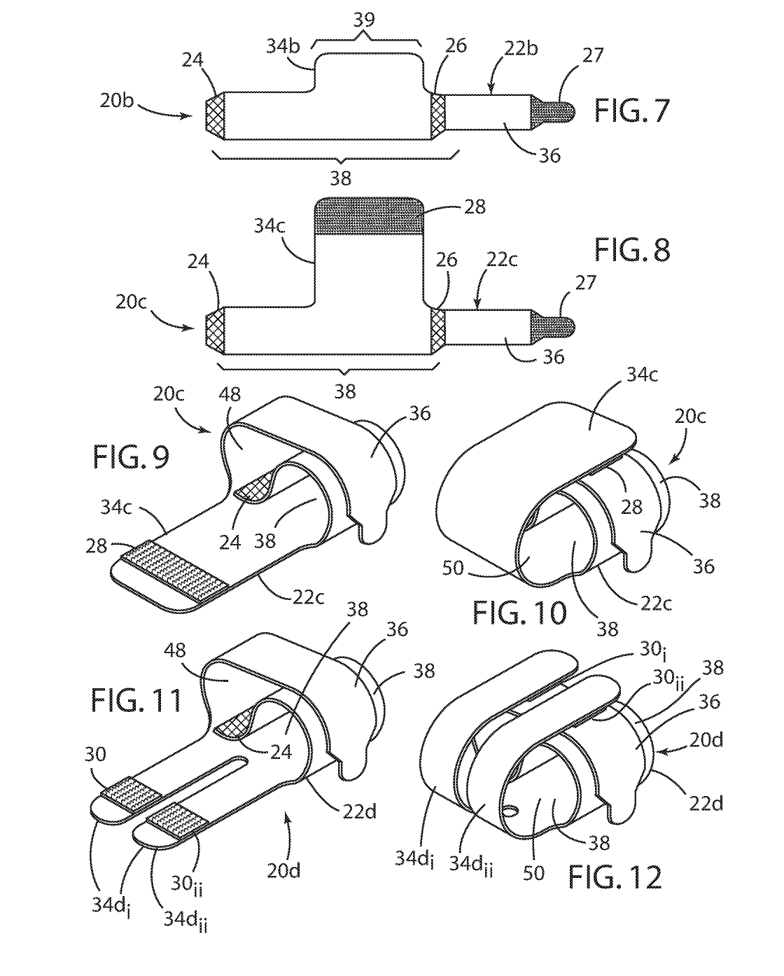

FIG. 7 illustrates another approach 20b where the extended toe pad 34b extends further beyond body portion 22b. This extension would be more likely a 10 mm to 20 mm extension beyond body portion 22b which forms loop 38. Dimension 39 presents the area for placement under the end of the user's toes.

FIG. 8 illustrates another approach 20c to the present embodiments by further extending toe pad 34c even further to not only extend past the user's toe but far enough to wrap upwards and back around the user's toes rearward in a longitudinal direction of the user's foot forming a third loop 50 (FIG. 10). In this embodiment, toe pad 34c at its distal end preferably has a second hook fastener 28 to attach to an exterior portion of strap 36, said exterior portion of strap 36 also having a loop surface as described above for the exterior surface 44 of loop 38. FIG. 9 shows the embodiment of FIG. 8 as it would initially appear while being applied to a user's foot with the second toe extending through the first loop 38 and the third toe extending through the second loop 48. The additional step of wrapping the extended toe pad 34 up and over the toes is shown in FIG. 10 forming the third loop 50.

FIGS. 11-12 show a slight variation of the embodiment of FIGS. 8-10. In this embodiment 20d, toe pad 34d has a flap for each toe (34di and 34dii) for added flexibility. Each flap 34d has a hook portion 30 to variably and releasably fasten and adjust loop 50.

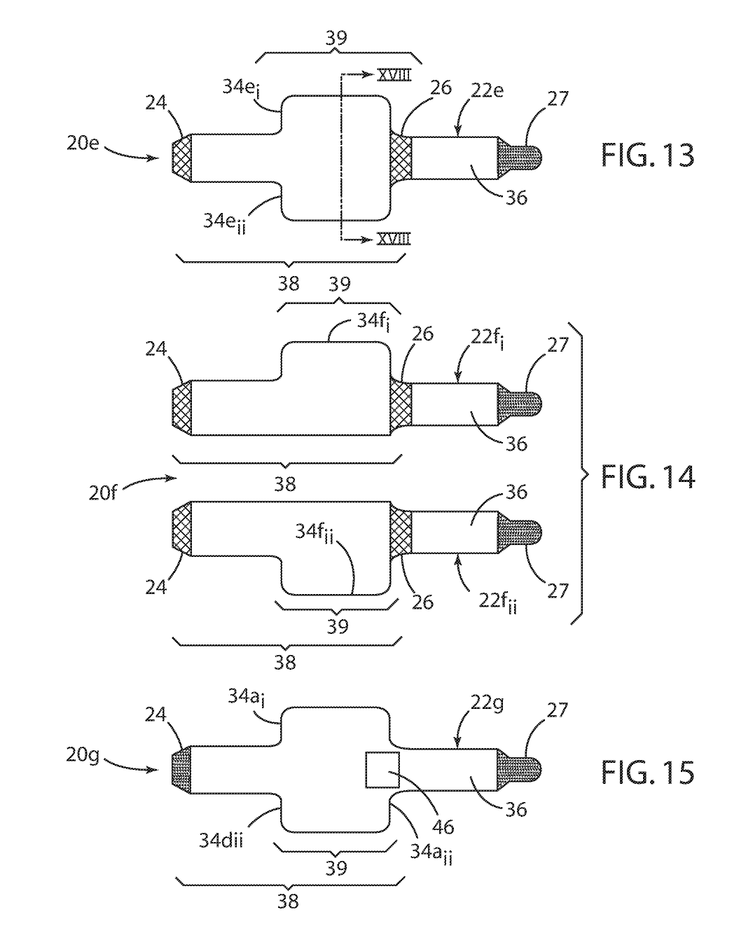

FIG. 13 illustrates another approach 20e to the present embodiments. In this embodiment, extended pads (34ei and 34eii) extend along both sides the longitudinal axis of body 22e. This allows a user to not have to differentiate between a left or right foot configured toe wrap since one of the extended pad 34 would always be extending beyond the user's toes. Additional benefits are also realized with this embodiment. A user would not have to purchase a set of toe wraps, but could buy a single toe wrap if toe deformities are only found on one foot. Also, the pad 34 not directed under the user's toes would extend rearwardly toward the user's ball-of-foot region. This additional padding may reduce pain associated with metatarsalgia—particularly the second toe (in association with a condition called second metatarsophalangeal stress syndrome). FIG. 18 shows a cross section of toe wrap 20e of FIG. 13 taken along section line XVIII-XVIII. As shown, body 22e has a loop layer 44 for the exterior surface, a foam layer 42, and an interior layer 40. Interior layer 40 in some embodiments can also be a loop layer as discussed above. In other embodiments layer 40 can be a moisture or odor barrier.

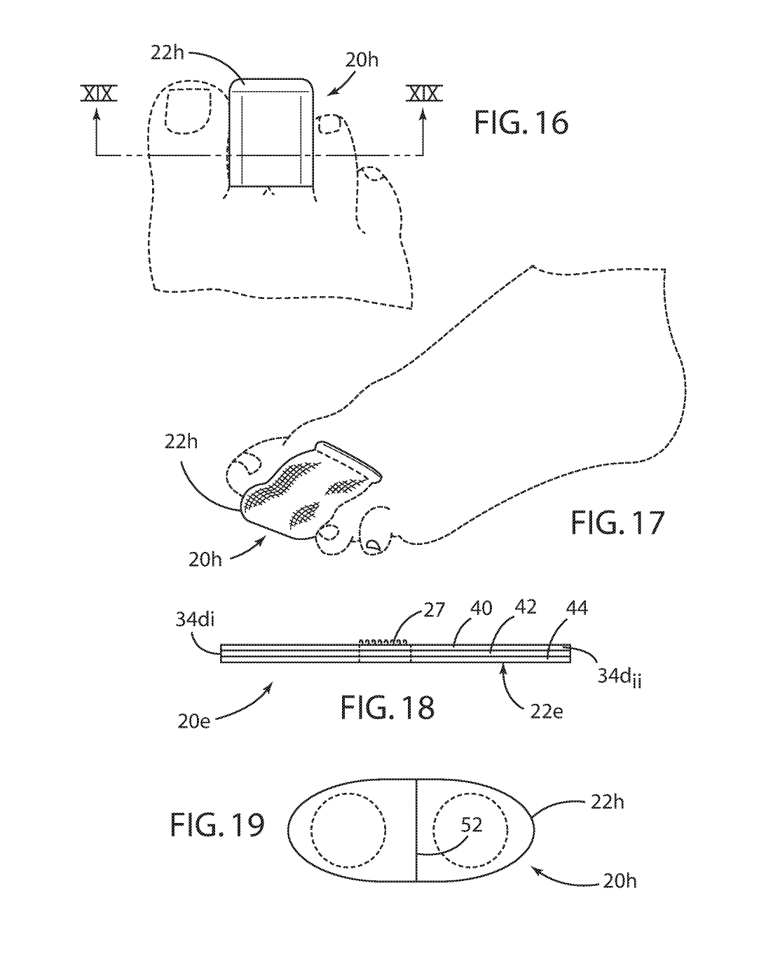

Other approaches are also possible within the scope of the preferred embodiments. FIG. 14 illustrates a pair of mirror image (left/right) toe wraps 20f, which can be sold as a kit. The embodiment shown is similar to the embodiment illustrated in FIG. 7, though it is noted that any of the presented embodiments can be paired in this way. FIG. 15 shows an embodiment 20g, where the toe loop 38 is not formed by heat seal during manufacture. In this embodiment portions 24 and 46 cooperate to form a hook and loop fastener. Portion 46 can be applied to either side of body 20g so that the loop can be formed exposing either of the body sides. In this instance, both surfaces of the body would need to be a loop surface to allow hook attachment 24. Hook 24 would also need to be applied to each side of body 20 at portion 24. Also, in FIGS. 16-17 toe wrap 20h is a sheath covering two toes, which may or may not have a flexible partition such as shown in FIG. 19.

It will be understood that various changes in the details, materials, and arrangements of the flexible package, the laminations, and methods, which have been herein described and illustrated in order to explain the nature of the package and methods, may be made by those skilled in the art within the principle and scope of the embodied packages, laminates and methods as expressed in the appended claims.

I claim:

1. A device to restrain at least one toe in a predetermined position for an extended period of time; the device comprising:
a flexible body composition forming a first loop at a first end, said first loop having a diameter sized to at least allow a toe to extend therethrough;
a second end of said body forming a strap having a distal end having a fastener to attach to an exterior surface of said first loop; and
wherein said first loop is configured to extend distally beyond an end of the toe to form a toe pad.

2. The device of claim 1, wherein the fastener is a hook and loop fastener.

3. The device of claim 1, wherein the device is formed from a single piece of material and wherein the first loop is formed by heat sealing the first end to a mid-portion of the body.

4. The device of claim 1, wherein the toe pad extends to allow covering the toe in a direction longitudinal to the foot, the toe pad having a distal end having a fastener to fasten to the exterior surface of the device.

5. The device of claim 4, wherein the toe pad is split to form two straps, on for each toe.

6. The device of claim 1, wherein said first loop is configured to extend proximally behind the toe to the ball of the foot region.

7. The device of claim 1, wherein the first loop is configured to apply to a deformed toe and allowing the distal end to extend under and around an adjacent toe, the distal end fastenable to the exterior surface of the first loop, thereby forming a second loop.

8. The device of claim 1, wherein the flexible body is a laminate comprising a soft exterior layer, a foam inner layer and a moisture resistant interior layer.

9. A method of restraining at least one toe in a predetermined position for an extended period of time; the method comprising the steps of:
inserting a first toe into a first loop of a flexible body first loop at a first end, said first loop having a diameter sized to at least allow a toe to extend therethrough and said first loop is configured to extend distally beyond an end of the toe to form a toe pad;
wrapping under and around an adjacent toe a second end of said flexible body forming a strap having a distal end having a fastener to attach to an exterior surface of said first loop; and
attaching the fastener to the exterior surface of the first loop.
